(19) United States
(12) Patent Application Publication
(10) Pub. No.: US 2023/0229054 A1
(43) Pub. Date: Jul. 20, 2023

(54) ELECTROPHORESIS SYSTEM, ELECTROPHORESIS APPARATUS, AND ELECTROPHORESIS ANALYSIS METHOD

(71) Applicant: SHIMADZU CORPORATION, Kyoto-shi (JP)

(72) Inventors: Kota OGINO, Kyoto-shi (JP); Akira HARADA, Kyoto-shi (JP); Kazunori SHIMIZU, Kyoto-shi (JP); Takashi MORIMOTO, Kyoto-shi (JP)

(73) Assignee: SHIMADZU CORPORATION, Kyoto-shi (JP)

(21) Appl. No.: 18/094,528

(22) Filed: Jan. 9, 2023

(30) Foreign Application Priority Data
Jan. 19, 2022 (JP) 2022-006743

(51) Int. Cl. G02F 1/167; G01N 27/447; G02F 1/1334; G16B 40/10

(52) U.S. Cl. CPC G02F 1/167; G01N 27/44721; G01N 27/44778; G01N 27/44743; G02F 1/13345; G16B 40/10

(57) ABSTRACT

This electrophoresis system includes an electrophoresis apparatus, an analysis apparatus, and a display unit. The analysis apparatus is configured to switch between a detailed display state in which a plurality of analysis result check displays including at least the gel image display are displayed in a result display area of the display unit and an enlarged display state in which only the gel image display among the plurality of analysis result check displays is enlarged and displayed in the result display area of the display unit.

### CROSS-REFERENCE TO RELATED APPLICATIONS

The related application number JP2022-006743, an electrophoresis system, an electrophoresis apparatus, an electrophoresis analysis method, and an electrophoresis analysis program, filed Jan. 19, 2022, by Kota Ogino, Akira Harada, Kazunori Shimizu, and Takashi Morimoto upon which this patent application is based are hereby incorporated by reference.

### BACKGROUND OF THE INVENTION

### Field of the Invention

The present invention relates to an electrophoresis system, an electrophoresis apparatus, and an electrophoresis analysis method.

### Background Art

In the related art, an analysis apparatus for separation data obtained by electrophoresis analysis has been known. Such an apparatus is disclosed in, for example, JP-A-2020-106351.

The analysis apparatus described in JP-A-2020-106351 analyzes separation data acquired by electrophoresis analysis by an electrophoresis apparatus. In the analysis of separation data acquired by electrophoresis analysis, components contained in a sample-to-be-analyzed are identified, by comparing reference data, which is separation data for a sample for reference containing known components with data-to-be-analyzed, which is separation data for the sample-to-be-analyzed. The analysis apparatus described in JP-A-2020-106351 acquires separation data acquired by electrophoresis analysis by the electrophoresis apparatus. Then, this analysis apparatus specifies the separation index value of each component peak in the separation data, and determines whether or not the respective component peaks of the reference data and the data-to-be-analyzed in the separation data are identical to each other. Further, the analysis apparatus described in JP-A-2020-106351 displays the gel images of data-to-be-analyzed and reference data side by side on a display device so as to make it easier to view the component peaks present in the data-to-be-analyzed. In this gel image, a band pattern indicating the separation index value of each component peak separated by electrophoresis is displayed.

Here, although it is not specified in JP-A-2020-106351, in general, when analyzing separation data acquired by electrophoresis analysis, an indication indicating the position of the well in which the sample that is the object-to-be-measured is placed, and waveforms of separation data (measurement values) acquired by the electrophoresis apparatus, and the like are displayed on the display device (display unit) together with the gel image. Therefore, since the size of the area where the gel image is displayed in the display area of the display device is limited, when there are a plurality of bands with similar values in the band pattern of the displayed gel image, the plurality of bands in the gel image are overlapped and displayed. In this case, even if the displayed gel image is checked, it is difficult to determine whether the separation is performed correctly in the electrophoresis analysis. Therefore, it is desired to improve the visibility of the distribution (band pattern) of the components separated by electrophoresis in the gel image display.

### SUMMARY OF THE INVENTION

The present invention has been made to solve the above problems, and one object of the present invention is to provide an electrophoresis system, an electrophoresis apparatus, and an electrophoresis analysis method capable of improving the visibility of the distribution of the components separated by electrophoresis in the gel image display.

In order to achieve the above object, an electrophoresis system according to a first aspect of the present invention includes: an electrophoresis apparatus including a measurement unit that measures an object-to-be-measured separated by electrophoresis in a channel including a separation channel for separating the object-to-be-measured; an analysis apparatus that analyzes components of the object-to-be-measured separated by electrophoresis, based on a measurement value of the object-to-be-measured that is measured by the measurement unit; and a display unit that displays, in a result display area, a plurality of analysis result check displays including at least a gel image display showing a distribution of the components of the object-to-be-measured analyzed by the analysis apparatus, in which the analysis apparatus is configured to switch between a detailed display state in which the plurality of analysis result check displays including at least the gel image display are displayed in the result display area of the display unit and an enlarged display state in which only the gel image display among the plurality of analysis result check displays is enlarged and displayed in the result display area of the display unit.

An electrophoresis apparatus according to a second aspect of the present invention includes: a measurement unit that measures an object-to-be-measured separated by electrophoresis in a channel including a separation channel for separating the object-to-be-measured, in which the electrophoresis apparatus is configured to switch between a detailed display state in which the plurality of analysis result check displays including at least a gel image display showing a distribution of the component of the object-to-be-measured analyzed based on the measurement value of the object-to-be-measured that is measured by the measurement unit are displayed in the result display area of the display unit and an enlarged display state in which only the gel image display among the plurality of analysis result check displays is displayed in the result display area of the display unit.

An electrophoresis analysis method according to a third aspect of the present invention includes: a step of analyzing a component of an object-to-be-measured separated by electrophoresis, based on a measurement value obtained by measuring the object-to-be-measured separated by electrophoresis in a channel including a separation channel for separating the object-to-be-measured; and a step of switching between a detailed display state in which the plurality of analysis result check displays including at least a gel image display showing a distribution of the analyzed component of the object-to-be-measured are displayed in the result display area of the display unit and an enlarged display state in which only the gel image display among the plurality of analysis result check displays is displayed in the result display area of the display unit.

In the electrophoresis system according to the first aspect, the electrophoresis apparatus according to the second aspect, and the electrophoresis analysis method according to the third aspect, a detailed display state in which the plurality of analysis result check displays including at least the gel image display are displayed in the result display area of the display unit and an enlarged display state in which only the gel image display among the plurality of analysis result check displays is enlarged and displayed in the result display area of the display unit are switched. Thus, a state in which the plurality of analysis result check displays are displayed in the result display area of the display unit and a state in which only the gel image display is displayed in the result display area of the display unit are switched, by switching between the detailed display state and the enlarged display state. Therefore, in a case where there are a plurality of bands with similar values in the band pattern of the gel image display in the detailed display state, the gel image display can be enlarged by switching to the enlarged display state. As a result, it is possible to improve the visibility of the distribution of the components separated by electrophoresis in the gel image display.

### BRIEF DESCRIPTION OF THE DRAWINGS

FIG. 1 is a block diagram showing the overall configuration of an electrophoresis system according to the present embodiment.

FIG. 2 is a schematic diagram for explaining the configuration of an electrophoresis apparatus of the present embodiment.

FIG. 3 is a diagram for explaining the configuration of a chip provided with a channel for electrophoresis.

FIG. 4 is a diagram showing an example of measurement values acquired by measurement of a measurement unit.

FIG. 5 is a diagram showing an example of display on the display unit in a detailed display state.

FIG. 6 is a diagram showing an example of display on the display unit in an enlarged display state.

FIG. 7 is a diagram (flow chart) for explaining an electrophoresis analysis method according to an embodiment.

### DETAILED DESCRIPTION OF THE INVENTION

Hereinafter, an embodiment embodying the present invention will be described with reference to the drawings.

Overall Configuration of Electrophoresis System

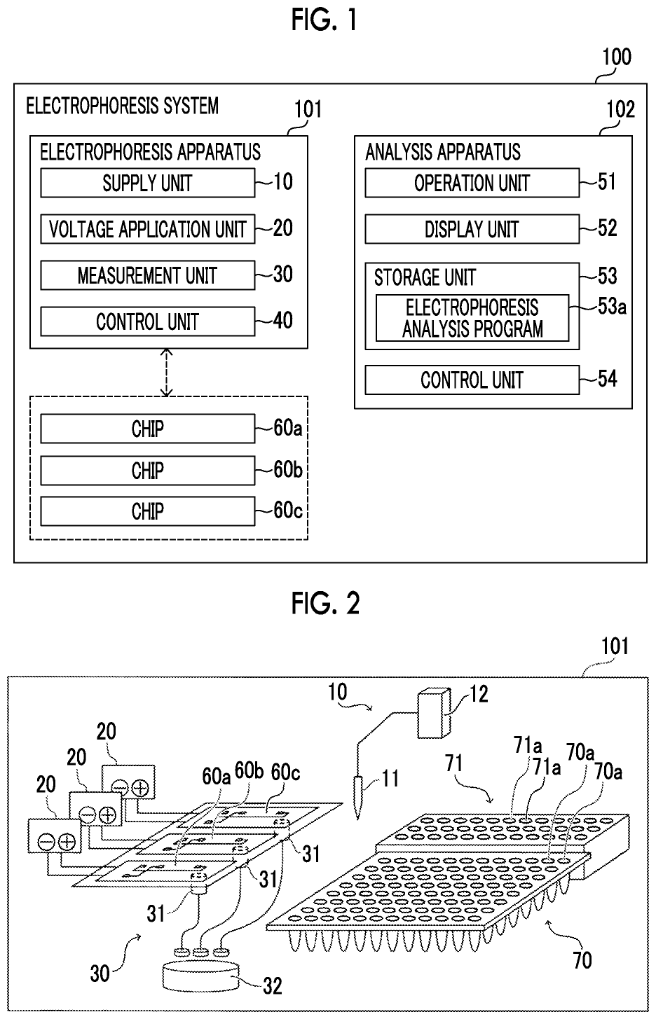

An electrophoresis system 100 according to an embodiment of the present invention is described with reference to FIGS. 1 to 6.

As shown in FIG. 1, an electrophoresis system 100 according to the present embodiment includes an electrophoresis apparatus 101 and an analysis apparatus 102.

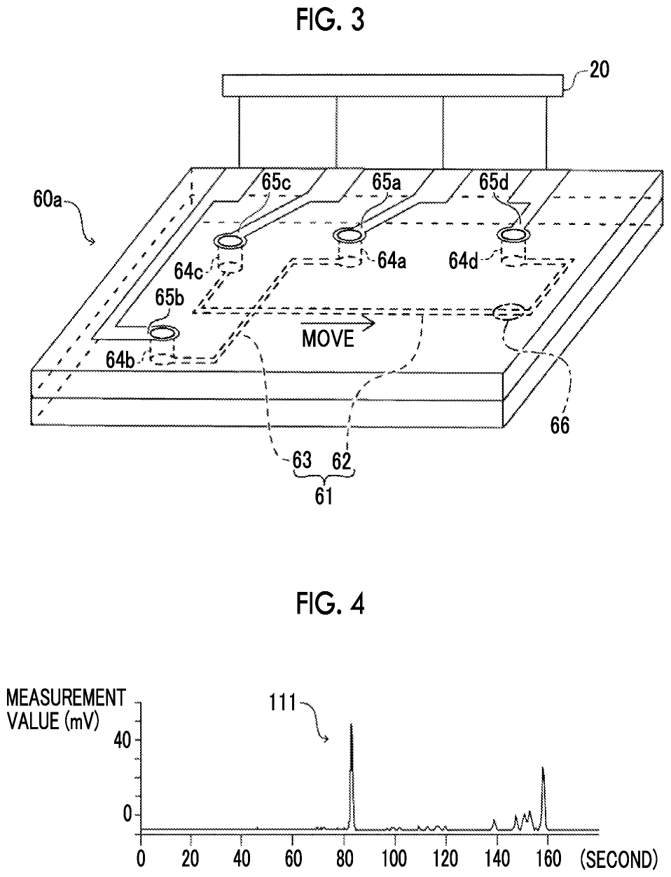

The electrophoresis apparatus 101 separates the object-to-be-measured by electrophoresis by using three chips 60a, 60b, and 60c, thereby measuring components contained in the object-to-be-measured. Specifically, in the electrophoresis apparatus 101, the objects-to-be-measured that are placed in advance on a plate 70 and a sample placement unit 71 (see FIG. 2) are separated by electrophoresis in the channel 61 (see FIG. 3) provided in each of the chips 60a to 60c. Then, the electrophoresis apparatus 101 measures the degree of separation (the degree of distribution of each component) of the object-to-be-measured separated by electrophoresis.

Configuration of Electrophoresis Apparatus

As shown in FIGS. 1 and 2, the electrophoresis apparatus 101 includes a supply unit 10, a voltage application unit 20, a measurement unit 30, and a control unit 40.

In the electrophoresis apparatus 101, the object-to-be-measured and the separation buffer are supplied to the channel 61 of each of the chips 60a, 60b, and 60c by the operation of the supply unit 10 in order to perform measurement by electrophoresis.

Objects-to-be-measured include, for example, deoxyribonucleic acid (DNA), ribonucleic acid (RNA), or protein. The object-to-be-measured includes a sample-to-be-measured (sample) for which the degree of separation of each component by electrophoresis is measured, and a reference sample (size standard) that is a reference for the measurement by electrophoresis of the sample-to-be-measured. The sample-to-be-measured is an object-to-be-measured whose degree of separation by electrophoresis, which is the measurement value 111 (see FIG. 4) measured by the measurement unit 30, is unknown. The reference sample is an object-to-be-measured containing nucleic acids or proteins whose separation characteristics such as molecular weight (chain length) have already been determined. That is, the sample-to-be-measured is an object-to-be-measured with unknown components, and the reference sample is an object-to-be-measured with known components.

Further, the object-to-be-measured is placed on the plate 70 and the sample placement unit 71. The plate 70 is provided with a plurality of wells 70a, which are a plurality of placement positions in which the objects-to-be-measured are placed. For example, the plate 70 is provided with 96 wells 70a arranged in a grid pattern of 8×12. The plate 70 is placed at a plate placement position inside the electrophoresis apparatus 101 by the operator with a plurality of types of objects-to-be-measured placed in all or part of each of the plurality of wells 70a. The object-to-be-measured is placed in the sample placement unit 71 separately from the plate 70. Further, the sample placement unit 71 has wells 71a, which are a plurality of placement positions in which objects-to-be-measured are placed. The wells 71a are arranged in a grid pattern of 3×12.

The separation buffer is a separation medium that fills the channel 61 (see FIG. 3) of each of the chips 60a, 60b, and 60c, before the object-to-be-measured is supplied. The separation buffer includes, for example, at least one of a pH buffer and a water-soluble polymer (cellulosic polymer, or the like). Further, the separation buffer is filled in a buffer container (not shown). Note that the separation buffer may be placed on the plate 70 or the sample placement unit 71. In the electrophoresis apparatus 101, an object-to-be-measured is supplied and electrophoresis is performed in a state in which the channel 61 is filled with a separation buffer in advance.

As shown in FIG. 2, the supply unit 10 has a probe 11 and a pump 12. By moving the probe 11, the supply unit 10 supplies the separation buffer and the object-to-be-measured (sample-to-be-measured and reference sample) placed on the plate 70 or the sample placement unit 71 to the chips 60a to 60c. The pump 12 adjusts the pressure for aspiration and discharge of the separation buffer and the object-to-be-measured in the probe 11.

As shown in FIG. 3, the chips 60a to 60c are each provided with a channel 61 therein. Here, the chips 60a, 60b and 60c have the same configuration. In the following description, the details of the chip 60a will be illustrated and described, and the chips 60b and 60c are the same as the chip 60a, so that the description of thereof will be omitted.

The chip 60a is a microchip for electrophoresis in which a channel 61 for performing electrophoresis is provided inside a pair of combined flat plate-like members. The channel 61 includes a separation channel 62 and a preparation channel 63. The separation channel 62 and the preparation channel 63 are provided so as to cross each other. The separation channel 62 is provided to separate the object-to-be-measured by electrophoresis. Further, the preparation channel 63 is provided to guide the object-to-be-measured to the separation channel 62.

At both ends of the preparation channel 63, reservoir portions 64a and 64b, which are spaces for supply and aspiration of the separation buffer and the object-to-be-measured, are provided. Similarly, both ends of the separation channel 62 are provided with reservoir portions 64c and 64d. Electrodes 65a and 65b are disposed in the reservoir portions 64a and 64b provided at both ends of the preparation channel 63, respectively. Electrodes 65c and 65d are disposed in the reservoir portions 64c and 64d provided at both ends of the separation channel 62, respectively.

In the electrophoresis apparatus 101, electrophoresis is performed by applying a voltage from the voltage application unit 20 to the plurality of electrodes 65a to 65d provided in the channel 61. Further, the magnitude of the voltage applied to the electrodes 65a to 65d is controlled by the control unit 40. Three voltage application units 20 (see FIG. 2) are provided to correspond to the respective chips 60a to 60c so as to apply a DC voltage to the channels 61 of each of the chips 60a to 60c. That is, similar to the chip 60a, with respect to the chips 60b and 60c, a DC voltage is applied to the channel 61 by the voltage application unit 20.

In the electrophoresis apparatus 101, in a case where measurement by electrophoresis is performed in the chip 60a, first, the supply unit 10 fills the entire interior of the channel 61 (the separation channel 62 and the preparation channel 63) with the separation buffer. Then, the supply unit 10 supplies the object-to-be-measured aspirated from, for example, a predetermined well 70a of the plate 70 to the reservoir portion 64a of the preparation channel 63. Then, by applying a predetermined voltage to the electrodes 65a to 65d by the voltage application unit 20, the object-to-be-measured moves inside the preparation channel 63 to a position where the preparation channel 63 and the separation channel 62 intersect. After that, by changing the magnitude of the voltage applied from the voltage application unit 20 to each of the electrodes 65a to 65d, the object-to-be-measured moves toward the electrode 65d (reservoir portion 64d) while being separated by electrophoresis inside the separation channel 62.

At this time, in separation measurement by electrophoresis, the object-to-be-measured moves inside the separation channel 62 at a different speed for each component contained in the object-to-be-measured, depending on the separation characteristics such as the molecular weight (chain length) of the contained component. In the electrophoresis apparatus 101, the separation characteristics of each component of the object-to-be-measured are measured by measuring the components that sequentially reach the measurement position 66 in the separation channel 62. In this manner, in the electrophoresis apparatus 101, the components contained in the object-to-be-measured are measured for each degree of separation (degree of migration).

As shown in FIG. 2, the measurement unit 30 measures an object-to-be-measured separated by electrophoresis in the channel 61 of each of the plurality (three) of chips 60a to 60c. For example, the measurement unit 30 performs fluorescence detection on the component of the object-to-be-measured that has been separated by electrophoresis. The measurement unit 30 has an LED 31 (light emitting diode) that emits excitation light to a measurement position 66 (see FIG. 3) of the separation channel 62. By applying excitation light from the LED 31 to each component of the object-to-be-measured moving in the separation channel 62 while being separated by electrophoresis, each component of the object-to-be-measured is excited and emits fluorescence. The measurement unit 30 measures the components of the object-to-be-measured separated by electrophoresis of the object-to-be-measured, by measuring this fluorescence with the photomultiplier tube 32 via, for example, an optical fiber and a filter member.

As shown in FIG. 4, the photomultiplier tube 32 outputs a measurement signal indicating a measurement value 111 to the control unit 40, according to the detected fluorescence intensity. The measurement value 111 based on the measurement by the measurement unit 30 shows a large value (peak) at the timing when the object-to-be-measured moving while being separated by electrophoresis passes the measurement position 66 (see FIG. 3). Thus, the amount (concentration) and composition (size) are analyzed as the degree of distribution of each component contained in the object-to-be-measured, based on the peak size and position (timing) of each component contained in the object-to-be-measured.

Note that the electrophoresis apparatus 101 is provided with a cleaning mechanism (not shown). The electrophoresis apparatus 101 cleans each part including the chips 60a to 60c and the supply unit 10 each time one object-to-be-measured is measured. The electrophoresis apparatus 101 is configured to repeatedly perform measurements using each of the chips 60a to 60c a plurality of times by cleaning the object-to-be-measured and the separation buffer remaining in the channel 61 with the cleaning mechanism. In this manner, the electrophoresis apparatus 101 sequentially measures each of the plurality of objects-to-be-measured placed in the plurality of wells 70a and 71a.

The control unit 40 controls the operation of each unit of the electrophoresis apparatus 101. The control unit 40 is, for example, a microcomputer (microcontroller) having a processing device such as a central processing unit (CPU) and a storage device such as a flash memory. Further, the control unit 40 includes a communication module and is configured to be able to communicate with the analysis apparatus 102. Then, based on the drive signal from the analysis apparatus 102, the control unit 40 controls the operation of each unit of the electrophoresis apparatus 101 so as to sequentially perform measurement by electrophoresis on a plurality of objects-to-be-measured placed on the plate 70 and the sample placement unit 71.

Specifically, based on the drive signal from the analysis apparatus 102, the control unit 40 operates the supply unit 10 to sequentially supply, for example, the objects-to-be-measured placed in the wells 70a of the plate 70 such that one type is measured each time on each of the chips 60a to 60c. Then, the control unit 40 causes the voltage application units 20 to apply a voltage to the channels 61 of the chips 60a to 60c, thereby separating (moving) the object-to-be-measured by electrophoresis. Further, the control unit 40 acquires the measurement values 111 measured by the measurement unit 30 provided corresponding to each of the chips 60a to 60c. Then, the control unit 40 acquires the measurement value 111, for each of the plurality of wells 70a of the plate 70 and each of the plurality of wells 71a of the sample placement unit 71. Then, the control unit 40 outputs the measurement value 111 of the object-to-be-measured that is measured by the measurement unit 30, for each of the chips 60a to 60c, to the analysis apparatus 102.

Configuration of Analysis Apparatus

As shown in FIG. 1, the analysis apparatus 102 includes an operation unit 51, a display unit 52, a storage unit 53, and a control unit 54. The analysis apparatus 102 is a computer for analyzing the components of the object-to-be-measured separated by electrophoresis, based on the measurement value 111 of the object-to-be-measured that is measured by the electrophoresis apparatus 101. The analysis apparatus 102 is configured to be able to communicate with the electrophoresis apparatus 101, and is configured to acquire the measurement values 111 acquired by the electrophoresis apparatus 101.

The operation unit 51 receives an input operation by the operator. Further, the operation unit 51 outputs an operation signal based on the received input operation to the control unit 54. The operation unit 51 is, for example, a keyboard and a pointing device such as a mouse.

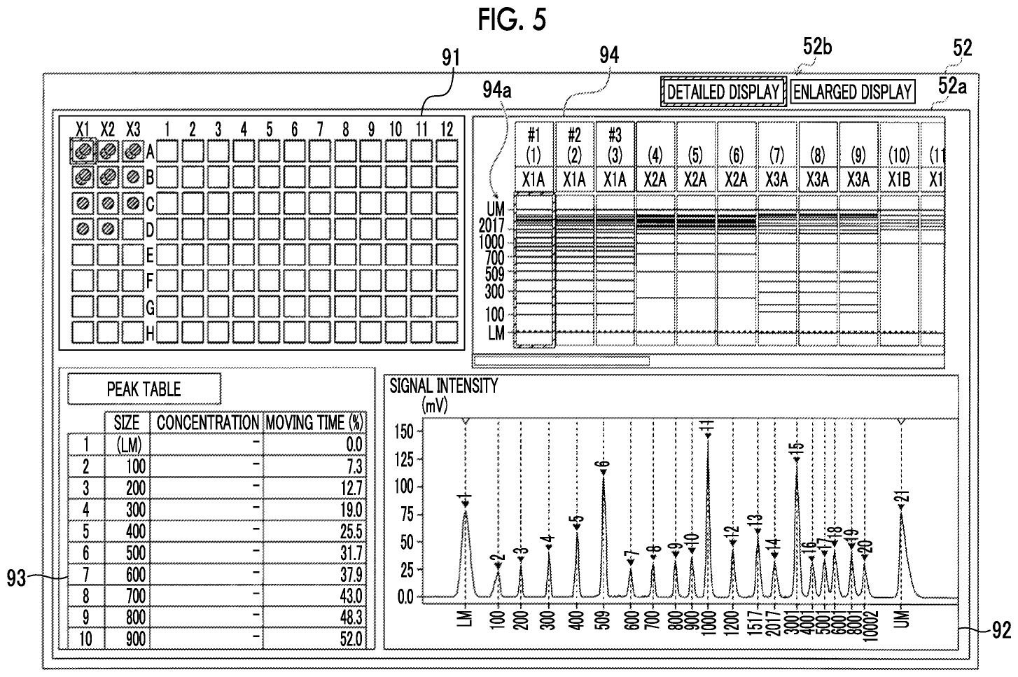

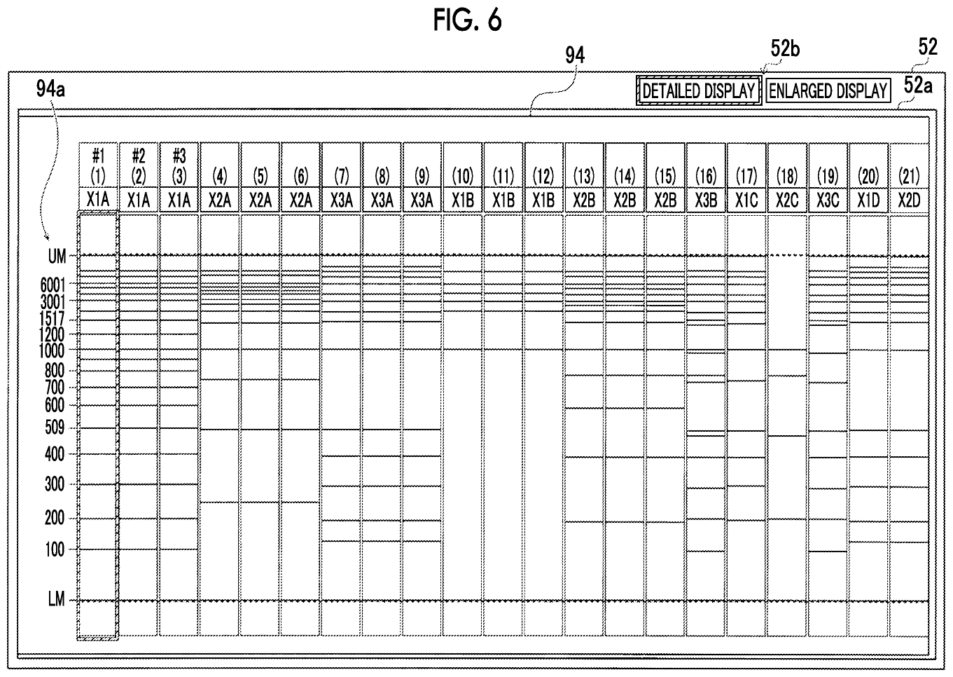

The display unit 52 is, for example, a monitor such as a liquid crystal display. The display unit 52 displays the information that is input under the control of the control unit 54. In addition, the display unit 52 displays the analysis result of the object-to-be-measured by the control unit 54 of the analysis apparatus 102. In the present embodiment, the display unit 52 displays a plurality of analysis result check displays in the result display area 52a (see FIGS. 5 and 6). The details of the display on the display unit 52 will be described later.

The storage unit 53 is configured by a storage device such as a hard disk drive or a Solid State Drive (SSD). The storage unit 53 stores the measurement values 111 acquired by the electrophoresis apparatus 101. The storage unit 53 also stores an electrophoresis analysis program 53a for operating the control unit 54. The storage unit 53 also stores various parameters such as preset setting values or setting values (measurement conditions) input by the operator.

The control unit 54 is a computer including a CPU, a Random Access Memory (RAM), a Read Only Memory (ROM), and the like. The control unit 54 executes control of each unit of the analysis apparatus 102 by executing a program (electrophoresis analysis program 53a) stored in the storage unit 53. Further, the control unit 54 is configured to be able to communicate with the control unit 40 of the electrophoresis apparatus 101 via a communication module (not shown).

Details of Control by Analysis Apparatus

The control unit 54 transmits drive signals for operating the electrophoresis apparatus 101 to the control unit 40. Specifically, based on an input operation received by the operation unit 51, the control unit 54 acquires various parameters for performing electrophoresis. For example, based on an input operation on the operation unit 51, the control unit 54 acquires well information indicating the wells 70a and 71a in which the objects-to-be-measured (reference sample and sample-to-be-measured) to be measured are placed, measurement condition information including information indicating the magnitude and time of the voltage to be applied, and the like, and schedule information indicating the measurement order of the objects-to-be-measured placed in the plurality of wells 70a and 71a. The well information, the measurement condition information, and the schedule information may be selected from the database stored in the storage unit 53 in advance. Then, the control unit 54 transmits drive signals including the acquired well information, measurement condition information, schedule information, or the like, to the control unit 40 of the electrophoresis apparatus 101. Then, the control unit 54 acquires the measurement value 111 acquired under the control by the control unit 40 based on the transmitted drive signal, from the control unit 40.

Then, as shown in FIGS. 5 and 6, the control unit 54 analyzes the object-to-be-measured separated by electrophoresis, based on the acquired measurement value 111. Then, the control unit 54 displays the analysis result of the object-to-be-measured, on the result display area 52a of the display unit 52. Specifically, the control unit 54 analyzes the size (separation index value) of each component of the object-to-be-measured separated by electrophoresis, based on the acquired measurement value 111. For example, in a case where the object-to-be-measured is DNA, the size to be analyzed is represented by the DNA chain length (the number of base pairs).

Switching Display

Further, in the present embodiment, the analysis apparatus 102 (the control unit 54) is configured to switch between the detailed display state and the enlarged display state, based on the operation received by the operation unit 51. Specifically, the analysis apparatus 102 displays a switching button display 52b on the upper side of the display unit 52 in a selectable manner. Then, the analysis apparatus 102 is configured to switch a display of the analysis result of the object-to-be-measured in the result display area 52a of the display unit 52 between the detailed display state and the enlarged display state, based on the operation unit 51 receiving the selection operation such as a click operation on the switching button display 52b.

Details of Detailed Display State

As shown in FIG. 5, in the detailed display state, the analysis apparatus 102 (control unit 54) is configured to display a plurality of analysis result check displays including a well position display 91, a measurement waveform display 92, a peak table 93, and a gel image display 94, in the result display area 52a of the display unit 52.

The well position display 91 indicates the position of each of the plurality of wells 70a and 71a in which each of the plurality of objects-to-be-measured are placed. In the well position display 91, the positions of the plurality of wells 70a and 71a are shown in a grid pattern so as to correspond to the arrangement of the wells 70a and 71a arranged in a grid pattern. For example, the positions of 96 wells 70a of 8×12 are indicated by squares (rectangles) arranged in a grid pattern of eight vertical columns A to H and 12 horizontal columns of 1 to 12. Further, the positions of the 3×12 wells 71a are indicated by squares (rectangles) arranged in a grid pattern of eight vertical columns A to H and three horizontal columns of X1 to X3.

Further, in the well position display 91, the wells 70a or 71a in which the objects-to-be-measured are placed are indicated by displaying a circle inside the squares arranged in a grid pattern. An indication in which two circles are overlapped indicates that the object-to-be-measured placed in the same well 70a or 71a is measured a plurality of times. Note that how the objects-to-be-measured (reference sample and sample-to-be-measured) are placed in the plurality of wells 70a and 71a is set based on the input operation on the operation unit 51 or the data stored in the storage unit 53.

Further, as shown in FIG. 5, the measurement waveform display 92 is a waveform (electropherogram) showing time-series values of the acquired measurement values 111. Specifically, the measurement waveform display 92 is represented by the size on the horizontal axis and the signal intensity (measurement value 111) measured by the measurement unit 30 on the vertical axis, based on the time-series values of the acquired measurement value 111. The measurement waveform display 92 also displays a numerical value indicating the size of the component of the separated object-to-be-measured.

Here, the object-to-be-measured separated by electrophoresis is mixed with an internal standard marker substance that is a reference in the analysis of the components of the object-to-be-measured. That is, in the measurement by electrophoresis, the channel 61 is supplied with an internal standard marker substance, which is a reference for the minimum value and the maximum value of the size (chain length) measured by electrophoresis, together with the object-to-be-measured. Specifically, the internal standard marker substance is placed in each of the wells 70a and 71a, in a state of being mixed with each object-to-be-measured. The internal standard marker substance has a Lower Marker (hereinafter referred to as LM) and an Upper Marker (hereinafter referred to as UM). LM is measured as a size of a sufficiently smaller value compared to the object-to-be-measured in the measurement by the measurement unit 30. Then, the UM is measured as a size of a sufficiently larger value compared to the object-to-be-measured in the measurement by the measurement unit 30. That is, the LM is sufficiently smaller in size than the reference sample and the sample-to-be-measured, and the UM is sufficiently larger in size than the reference sample and the sample-to-be-measured.

In the measurement by electrophoresis, common LM and UM are mixed for both the reference sample and the sample-to-be-measured. Then, based on the LM and UM measured in a case where the reference sample is measured and the LM and UM measured in a case where the sample-to-be-measured is measured, the size of the sample-to-be-measured is analyzed, by comparing the timings at which the measurement value 111 of the reference sample and the measurement value 111 of the sample-to-be-measured are measured. It is set in advance which of the plurality of objects-to-be-measured placed in the wells 70a and 71a is the reference sample. Further, in a case where a plurality of reference samples are placed in the well 70a or 71a, it is preset for each object-to-be-measured which reference sample is used for the analysis of the sample-to-be-measured.

Specifically, first, measurement by electrophoresis of a reference sample, which is a reference for the object-to-be-measured, is performed. Then, the analysis apparatus 102 detects a peak from the waveform of the measurement value 111 of the measured reference sample. Then, based on the detected LM, UM, and each peak of each component, with the size of LM is 0 and the size of UM is a predetermined value, a ratio between the time (timing) when the lower marker (LM) and the upper marker (UM) detected by the measurement unit 30 and the time (timing) when each component (peak) contained in the reference sample whose size is known in advance is detected by the measurement unit 30 is obtained, and a calibration curve is generated.

Then, as shown in FIG. 5, the analysis apparatus 102 (the control unit 54) analyzes the size of the sample-to-be-measured, based on the measurement value 111 acquired by electrophoresis in a state where a sample-to-be-measured, which is an object-to-be-measured of unknown size, is mixed with LM and UM and the generated calibration curve. Specifically, the analysis apparatus 102 detects peaks, from the waveform generated based on the measurement value 111 acquired by measuring a sample-to-be-measured of unknown size. Then, the analysis apparatus 102 detects LM and UM from the detected peaks, and analyzes the size corresponding to the peak from the calibration curve of the reference sample, based on the relative time ratio (moving time index) of the peaks included between LM and UM. Then, the analysis apparatus 102 displays the size corresponding to each detected peak, in the measurement waveform display 92. Further, the analysis apparatus 102 displays the specific numerical values of the sizes and moving time indices corresponding to the detected peaks in the peak table 93.

The gel image display 94 shows an analysis result (size) of each of the plurality of objects-to-be-measured. Specifically, in the gel image display 94, for each measurement of the object-to-be-measured using the chips 60a to 60c, an indication (image) showing the distribution of the component (size) of each of a plurality of objects-to-be-measured analyzed by the analysis apparatus 102 is displayed side by side as a plurality of analysis results. In the analysis result in the gel image display 94, a plurality of horizontal bars (band pattern, ladder) represent the size of each analyzed component of the object-to-be-measured. Further, in the analysis result in the gel image display 94, a plurality of horizontal bars are arranged at positions corresponding to the peaks of the waveform of the measurement value 111, according to size, with LM as the lower end and UM as the upper end. Further, in the gel image display 94, the position indicating the LM and the position indicating the UM are shown to be common positions in the plurality of analysis results displayed side by side. For example, in the analysis result in the gel image display 94, the magnitude of the measurement value 111 is represented by the shade of color, by setting the pixel value according to the magnitude of the measurement value 111 (signal intensity).

Further, the gel image display 94 displays a well number indicating the well 70a or 71a in which the object-to-be-measured corresponding to each analysis result is placed, and a measurement order number indicating the measurement order. Further, the analysis apparatus 102 is configured to display the chip number in the gel image display 94. The chip number is a number indication of any of 1 to 3 indicating each of the chips 60a to 60c. Then, in the gel image display 94, in the analysis of the measurement values 111 acquired by the measurements of each of the chips 60a to 60c, only the analysis result of the reference sample that is the reference for generating the calibration curve is indicated by the chip number.

Note that the analysis apparatus 102 (control unit 54) is configured to display, on the display unit 52, a measurement waveform display 92 and a peak table 93 corresponding to one analysis result selected from among a plurality of analysis results in the gel image display 94. Specifically, the operation unit 51 receives a selection operation of selecting one analysis result from images showing a plurality of analysis results displayed side by side in the gel image display 94. The analysis apparatus 102 is configured to display, on the display unit 52, a measurement waveform display 92 and a peak table 93 corresponding to one selected analysis result, based on a selection operation received by the operation unit 51. An identification display having a frame shape is displayed on the gel image display 94 such that the selected analysis result can be identified. Further, character information indicating the positions of the wells 70a and 71a corresponding to the selected analysis result, the type of object-to-be-measured (reference sample or sample-to-be-measured), and the number of the chips 60a to 60c used for measurement may be displayed on the display unit 52.

In the present embodiment, the analysis apparatus 102 (control unit 54) displays a scale 94a in the gel image display 94. The scales 94a represent the measure of separation of the separated components of the object-to-be-measured, in the analysis results displayed in the gel image display 94. Specifically, the scale 94a represents the size of each analyzed component of the object-to-be-measured. Further, numerical values corresponding to the scales 94a are displayed in the gel image display 94. The numerical value on the scale 94a represents the specific numerical value of the size of each component of object-to-be-measured. Further, the numerical values on the scales 94a are acquired based on the analysis of the components of the reference sample among the objects-to-be-measured. That is, the size (the size of the detected peak) of the component contained in the reference sample set for generating the calibration curve is acquired as a numerical value corresponding to the scale 94a.

In the present embodiment, the analysis apparatus 102 (control unit 54) is configured to reduce and thin out the number of displayed numerical values on the scales 94a such that the numerical values corresponding to the scales 94a in the gel image display 94 do not overlap each other. Further, the analysis apparatus 102 thins out the number of displayed numerical values, in a state in which the bias in the display intervals of the numerical values on the scales 94a is reduced. Specifically, in a case where the numerical values of the sizes of the reference sample are displayed on the scales 94a of the gel image display 94, when the numerical values overlap each other, the analysis apparatus 102 thins out the number of displayed numerical values on the scales 94a. At this time, in the numerical values corresponding to the scales 94a, the numerical values to be thinned out are set such that the intervals between the adjacent numerical values among the displayed numerical values are approximately equal in the vertical direction (such that the difference in the vertical direction does not increase).

Further, in the present embodiment, the analysis apparatus 102 (control unit 54) displays indications indicating the reference values (lower limit and upper limit) corresponding to the internal standard marker substance (LM and UM), regardless of the degree of thinning of the numerical values on the scales 94a in the gel image display 94. Specifically, the analysis apparatus 102 displays “LM” at the position corresponding to the size of LM and “UM” at the position corresponding to the size of UM, on the scales 94a. The indications of “UM” and “LM” are also displayed so as not to overlap the numerical values on the scales 94a with each other.

In addition, in the gel image display 94, when displaying a plurality of analysis results side by side by performing measurement a plurality of times, if the width of the display area in the arrangement direction (horizontal direction) is insufficient, the analysis results to be displayed are changed, by scrolling the display in the horizontal direction.

Details of Enlarged Display State

Further, as shown in FIG. 6, in the enlarged display state, the analysis apparatus 102 (control unit 54) is configured to enlarge and display only the gel image display 94 among the plurality of analysis result check displays, in the result display area 52a of the display unit 52, without displaying the well position display 91, the measurement waveform display 92, and the peak table 93.

In the present embodiment, in the gel image display 94, the analysis apparatus 102 (control unit 54) sets the display size in the arrangement direction (horizontal direction) of each of the plurality of analysis results to a common size in the detailed display state and the enlarged display state. Thus, the analysis apparatus 102 is configured to display a larger number of analysis results in the enlarged display state than in the detailed display state.

That is, in the enlarged display state, the analysis apparatus 102 is configured not to enlarge the horizontal size of the analysis result in the gel image display 94, but to enlarge only the vertical size. Therefore, in the enlarged display state, the number of displayed analysis results displayed as the gel image display 94 in the result display area 52a of the display unit 52 is greater than in the detailed display state. In addition, even in the enlarged display state, in a case where the width of the display area is insufficient, the analysis result to be displayed can be changed by scrolling the display in the horizontal direction.

In the present embodiment, the analysis apparatus 102 (control unit 54) is configured to make the degree of thinning of the numerical values on the scales 94a in the gel image display 94 different from each other, in the detailed display state and the enlarged display state. Specifically, the analysis apparatus 102 is configured to make the degree of thinning of the number of displayed numerical values on the scales 94a different from each other in the detailed display state and the enlarged display state such that the number of displayed numerical values is larger in the enlarged display state than in the detailed display state. In both the detailed display state and the enlarged display state, the display sizes of the displayed numerical values on the scales 94a are common size.

Further, the analysis apparatus 102 (control unit 54) is configured to thin out the number of displayed numerical values, in a state in which the bias in the display intervals of the numerical values displayed on the scales 94a is reduced, even in the enlarged display state. That is, in the present embodiment, when the detailed display state and the enlarged display state are switched, the analysis apparatus 102 is configured to change the degree of thinning of numerical values corresponding to the scales 94a, in a state in which the bias in the display of the numerical values displayed on the scales 94a is reduced.

Specifically, when the detailed display state is switched to the enlarged display state, the analysis apparatus 102 vertically enlarges the display (image) of the plurality of analysis results in the gel image display 94. At this time, the analysis apparatus 102 changes the degree of thinning of displayed numerical values on the scales 94a so as to increase the number of displayed numerical values, in a state in which the numerical values do not overlap each other and the bias in display intervals is reduced.

In addition, in the present embodiment, the analysis apparatus 102 (control unit 54), even in the enlarged display state, displays indications indicating the reference values (lower limit and upper limit) corresponding to the internal standard marker substance (LM and UM), regardless of the degree of thinning of the number of displayed numerical values on the scales 94a. That is, in both the detailed display state and the enlarged display state, the display of “LM” indicating the lower limit of size and the display of “UM” indicating the upper limit of size are displayed so as to indicate the reference on the scales 94a in the gel image display 94 (lower limit and upper limit).

Regarding Electrophoresis Analysis Method

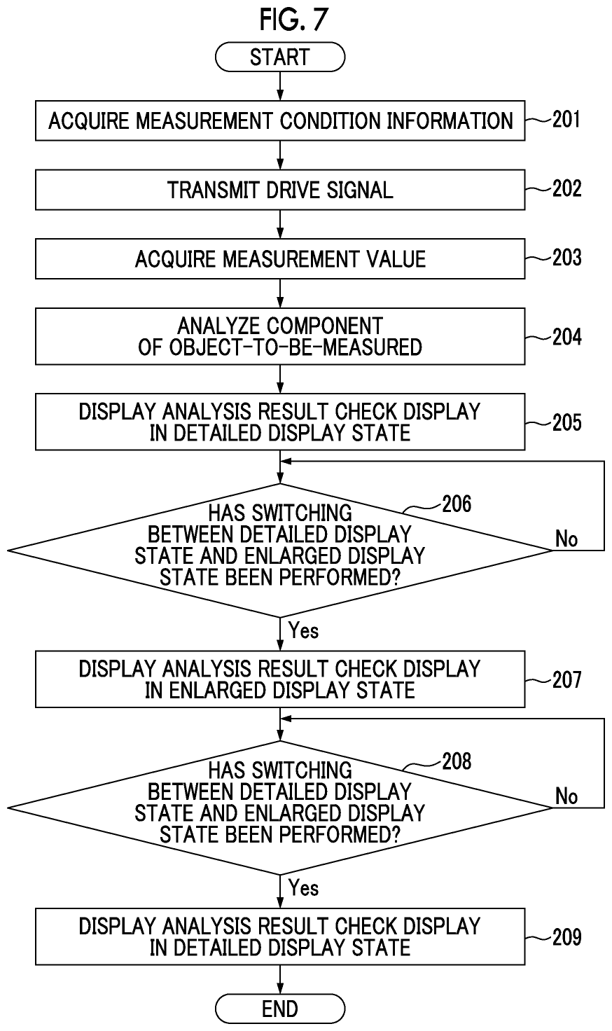

Next, an electrophoresis analysis method using the electrophoresis system 100 according to the present embodiment will be described with reference to FIG. 7. The control process in steps 201 to 209 is performed by executing the electrophoresis analysis program 53a stored in the storage unit 53 by the control unit 54 (analysis apparatus 102).

First, in step 201, measurement condition information for performing measurement is acquired. Specifically, well information indicating the wells 70a and 71a in which the objects-to-be-measured to be measured are placed, schedule information indicating the order of measurement, information indicating types of objects-to-be-measured (reference sample and object-to-be-measured) placed in the wells 70a and 71a, or the like are acquired (set) along with measurement condition information including information indicating the magnitude and time of the voltage applied by the voltage application unit 20. These pieces of information may be acquired based on an input operation on the operation unit 51 or may be acquired from information stored in advance in the storage unit 53 or the like.

Next, in step 202, a drive signal including the acquired measurement condition information is transmitted to the control unit 40 of the electrophoresis apparatus 101. The drive signal includes the acquired well information, schedule information, and the like, in addition to the measurement condition information. Based on this drive signal, in the electrophoresis apparatus 101, measurement by electrophoresis using the chips 60a to 60c is performed for each object-to-be-measured in the predetermined wells 70a and 71a in the set order.

Next, at step 203, the measurement value 111 is acquired based on the measurement by the measurement unit 30 of the electrophoresis apparatus 101.

Next, in step 204, an analysis based on the acquired measurement values 111 is performed. Specifically, based on the acquired measurement values 111, the component of the object-to-be-measured separated by electrophoresis is analyzed.

Next, in step 205, the analysis result check displays in the detailed display state are displayed in the result display area 52a of the display unit 52, based on the analysis of the component of the object-to-be-measured. Specifically, a plurality of analysis result check displays including the well position display 91, the measurement waveform display 92, the peak table 93, and the gel image display 94 are displayed in the result display area 52a of the display unit 52.

Next, in step 206, it is determined whether or not an input operation for switching between the detailed display state and the enlarged display state has been received. Specifically, based on the operation signal from the operation unit 51, it is determined whether or not an input operation for switching between the detailed display state and the enlarged display state has been received, by the selection operation on the switching button display 52b displayed on the display unit 52. When it is determined that the input operation for switching between the detailed display state and the enlarged display state has been received, the process proceeds to step 207. When it is not determined that the input operation for switching between the detailed display state and the enlarged display state has been received, the control process is on standby while the display in the detailed display state is continued.

In step 207, the detailed display state is switched to the enlarged display state, based on the input operation received by the operation unit 51. Specifically, in the result display area 52a of the display unit 52, the well position display 91, the measurement waveform display 92, and the peak table 93 are not displayed, and only the gel image display 94 is enlarged and displayed.

Next, in step 208, it is determined whether or not an input operation for switching again between the detailed display state and the enlarged display state has been received. Specifically, similar to step 206, based on the operation signal from the operation unit 51, it is determined whether or not an input operation for switching between the detailed display state and the enlarged display state has been received, by the selection operation on the switching button display 52b displayed on the display unit 52. When it is determined that the input operation for switching between the detailed display state and the enlarged display state has been received, the process proceeds to step 209. When it is not determined that the input operation for switching between the detailed display state and the enlarged display state has been received, the control process is on standby while the display in the enlarged display state is continued.

In step 209, the enlarged display state is switched again to the detailed display state, based on the input operation received by the operation unit 51. The display in the result display area 52a of the display unit 52 in the detailed display state is the same as in step 205.

After step 209, switching between the detailed display state and the enlarged display state may be performed again. Further, after the analysis of the component of the object-to-be-measured in step 204, the analysis result check display may be displayed in an enlarged display state instead of a detailed display state.

### Effect of Present Embodiment

In the present embodiment, the following effects can be obtained.

In the electrophoresis system 100 and the electrophoresis apparatus 101 of the present embodiment, as described above, a detailed display state in which the plurality of analysis result check displays including at least the gel image display 94 are displayed in the result display area 52a of the display unit 52 and an enlarged display state in which only the gel image display 94 among the plurality of analysis result check displays is enlarged and displayed in the result display area 52a of the display unit 52 are switched. Thus, a state in which the plurality of analysis result check displays are displayed in the result display area 52a of the display unit 52 and a state in which only the gel image display 94 is displayed in the result display area 52a of the display unit 52 are switched, by switching between the detailed display state and the enlarged display state. Therefore, in a case where there are a plurality of bands with similar values (sizes) in the band pattern of the gel image display 94 in the detailed display state, the gel image display 94 can be enlarged by switching to the enlarged display state. As a result, it is possible to improve the visibility of the distribution of the components separated by electrophoresis in the gel image display 94.

Further, in the above-described embodiment, further effects can be obtained by configuring as follows.

That is, in the present embodiment, as described above, the analysis apparatus 102 is configured to reduce and thin out a number of displayed numerical values corresponding to the scales 94a in the gel image display 94 such that the numerical values are not overlapped with each other, and make the degree of thinning of the number of displayed numerical values different from each other, in the detailed display state and the enlarged display state. With this configuration, in both the detailed display state and the enlarged display state, the numerical values on the scales 94a in the gel image display 94 are reduced and thinned out so as not to overlap each other, so that it is possible to reduce deterioration in the visibility of the numerical values on the scales 94a in the gel image display 94. Therefore, in the gel image display 94 in both the detailed display state and the enlarged display state, the visibility of the distribution of the separated components can be improved, and a specific numerical value indicating the separated component (size) can be easily checked.

Further, in the present embodiment, as described above, the analysis apparatus 102 is configured to display the numerical values in a common display size, in the detailed display state and the enlarged display state, and make the degree of thinning of the number of displayed numerical values different from each other such that the number of displayed numerical values in the enlarged display state is larger than in the detailed display state. With this configuration, the number of displayed numerical values on the scales 94a of the gel image display 94 increases in the enlarged display state, so that the visibility of the distribution of the separated component in the gel image display 94 in the enlarged display state can be improved, and a specific numerical value indicating the separated component (size) can be checked in detail. Further, since the numerical values on the scales 94a are displayed in a common display size in the detailed display state and the enlarged display state, the number of the displayed numerical values on the scales 94a in the detailed display state is not smaller. Therefore, it is possible to reduce deterioration in the visibility of numerical values in the detailed display state.

Further, in the present embodiment, as described above, the electrophoresis apparatus 101 is configured to separate each of the plurality of objects-to-be-measured by electrophoresis, and

the analysis apparatus 102 is configured to display, in the result display area 52a of the display unit 52, the gel image display 94 in which a plurality of analysis results showing the distribution of components of each of the plurality of objects-to-be-measured are displayed side by side, and

set a display size in an arrangement direction of each of the plurality of analysis results to a common size in the detailed display state and the enlarged display state such that more analysis results are displayed in the enlarged display state than in the detailed display state. With this configuration, by switching from the detailed display state to the enlarged display state, the visibility of the gel image display 94 can be improved, and the number of analysis results displayed in the enlarged display state is larger than in the detailed display state, so that more analysis results can be viewed as a list. Therefore, when comparing a plurality of analysis results, by switching from the detailed display state to the enlarged display state, more analysis results can be easily compared.

Further, in the present embodiment, as described above, the electrophoresis apparatus 101 is configured to separate, by electrophoresis, the object-to-be-measured mixed with an internal standard marker substance that serves as a reference in analysis of the component of the object-to-be-measured, and the analysis apparatus 102 is configured to display indications indicating the reference values (LM and UM) corresponding to the internal standard marker substance in the gel image display 94, in both the detailed display state and the enlarged display state, regardless of the degree of thinning of the number of displayed numerical values. With this configuration, even when switching between the detailed display state and the enlarged display state, the indications indicating the reference values (LM and UM) corresponding to the internal standard marker substances can be kept displayed in the gel image display 94. Therefore, even when the degree of thinning of the numerical values on the scales 94a in the gel image display 94 is changed by switching between the detailed display state and the enlarged display state, the indications indicating the reference values corresponding to the internal standard marker substance can be checked, so that the reference for the separated components can be readily checked, regardless of the size of the gel image display 94.

Further, in the present embodiment, as described above, the analysis apparatus 102 is configured to make the degree of thinning of the number of displayed numerical values, in the detailed display state and the enlarged display state different from each other, in a state where the bias in the display intervals of the numerical values in the gel image display 94 is reduced. With this configuration, in both the detailed display state and the enlarged display state, the bias in the numerical values on the scales 94a in the gel image display 94 is reduced, so that numerical values can be displayed so as to make the display intervals of numerical values on the scales 94a in the gel image display 94 substantially uniform. Therefore, in the gel image display 94, the degree of distribution of specific numerical values of the separated components (sizes) can be more easily grasped than when the numerical values on the scales 94a are biased and displayed.

Further, in the present embodiment, as described above, the electrophoresis apparatus 101 is configured to separate, by electrophoresis, each of a reference sample that is the object-to-be-measured with known components and a sample-to-be-measured that is the object-to-be-measured with unknown components, and the analysis apparatus 102 is configured to acquire the numerical values based on the analysis of the components of the reference sample, and make the degree of thinning of the number of displayed numerical values acquired based on the analysis of the components of the reference sample different from each other, in the detailed display state and the enlarged display state. With this configuration, it is possible to easily check which component of the reference sample with known components each separated component of the sample-to-be-measured with unknown components corresponds to, by checking the numerical values on the scales 94a in the gel image display 94. Therefore, by checking the numerical values on the scales 94a displayed on the gel image display 94, it is possible to easily check the specific numerical value indicating the separated component (size) of the sample-to-be-measured.

Further, in the present embodiment, as described above, the electrophoresis apparatus 101 is configured to separate each of the plurality of objects-to-be-measured by electrophoresis, and the analysis apparatus 102 is configured to, in the detailed display state, display the plurality of analysis result check displays including a measurement waveform display 92 showing time-series values of the measurement values 111, a well position display 91 showing a position of each of a plurality of wells 70a and 71a in which each of the plurality of objects-to-be-measured is arranged, and the gel image display 94 in which a plurality of analysis results showing a distribution of a component of each of the plurality of objects-to-be-measured are displayed side by side, in the result display area 52a of the display unit 52, and in the enlarged display state, enlarge and display only the gel image display 94 among the plurality of analysis result check displays, in the result display area 52a of the display unit 52, without displaying the measurement waveform display 92 and the well position display 91. With this configuration, in the detailed display state, the gel image display 94 including the analysis result of the component of the object-to-be-measured, the positions of the wells 70a and 71a where the objects-to-be-measured are arranged, and the waveform of the measurement value 111 can be checked, so that the details of the analysis of the separated object-to-be-measured can be checked. Therefore, by switching between the detailed display state and the enlarged display state, checking of the details of the analysis and checking of the gel image display 94 with improved visibility can be easily switched according to the situation.

Further, in the present embodiment, as described above, the electrophoresis system 100 includes the operation unit 51 that receives an input operation, and the analysis apparatus 102 is configured, in the detailed display state, display a plurality of analysis result check displays including the gel image display 94 in which a plurality of analysis results are displayed side by side, and the measurement waveform display 92 corresponding to one analysis result selected from among a plurality of analysis results in the gel image display 94 based on the operation received by the operation unit 51, in the result display area 52a of the display unit 52, and is configured to switch between the detailed display state and the enlarged display state, based on the operation received by the operation unit 51. With this configuration, an operator who analyzes the object-to-be-measured separated by electrophoresis can easily switch between the detailed display state and the enlarged display state by operating the operation unit 51. By selecting one analysis result in the gel image display 94 in the detailed display state, the operator can easily check the measurement waveform display 92 corresponding to the selected analysis result. As a result, checking the enlarged gel image display 94 in the enlarged display state, and checking the measurement waveform display 92 corresponding to one analysis result selected from the gel image display 94 in the detailed display state can be easily switched by performing an input operation on the operation unit 51.

Effects of Electrophoresis Analysis Method and Electrophoresis Analysis Program According to Present Embodiment

The electrophoresis analysis method and the electrophoresis analysis program 53a of the present embodiment can obtain the following effects.

In the electrophoresis analysis method and the electrophoresis analysis program 53a of the present embodiment, by being configured as described above, a detailed display state in which the plurality of analysis result check displays including at least the gel image display 94 are displayed in the result display area 52a of the display unit 52 and an enlarged display state in which only the gel image display 94 among the plurality of analysis result check displays is enlarged and displayed in the result display area 52a of the display unit 52 are switched. Thus, a state in which the plurality of analysis result check displays are displayed in the result display area 52a of the display unit 52 and a state in which only the gel image display 94 is displayed in the result display area 52a of the display unit 52 are switched, by switching between the detailed display state and the enlarged display state. Therefore, in a case where there are a plurality of bands with similar values (sizes) in the band pattern of the gel image display 94 in the detailed display state, the gel image display 94 can be enlarged by switching to the enlarged display state. As a result, it is possible to provide an electrophoresis analysis method and an electrophoresis analysis program 53a capable of improving the visibility of the distribution of components separated by electrophoresis in the gel image display 94.

### Modification Example

In addition, the embodiments disclosed here should be considered to be exemplary and not restrictive in all respects. The scope of the present invention is shown not by the description of the above embodiment but by the scope of claims, and further includes all changes (modification examples) within the meaning and scope equivalent to the scope of claims.

For example, in the above-described embodiment, an example is shown in which the analysis apparatus 102 that analyzes the component of the object-to-be-measured is provided separately from the electrophoresis apparatus 101, but the present invention is not limited to this. In the present invention, the electrophoresis apparatus 101 and the analysis apparatus 102 may be integrally formed. Similarly, only the display unit 52 may be formed integrally with the electrophoresis apparatus 101. Further, the display unit 52 may be placed separately while being spaced apart from both the electrophoresis apparatus 101 and the analysis apparatus 102.

Further, in the above-described embodiment, an example is shown in which the number of displayed numerical values is reduced and thinned out such that the numerical values corresponding to the scales 94a in the gel image display 94 do not overlap each other, but the present invention is not limited to this. For example, the numerical values on the scales 94a may be displayed on the scales 94a in a state in which the numerical values overlap within a recognizable range. Alternatively, the display size of the numerical values may be changed such that the numerical values on the scales 94a do not overlap each other, without changing the number of displayed values.

Further, in the above-described embodiment, an example is shown in which in the gel image display 94, the display size in the arrangement direction (horizontal direction) of each of the plurality of analysis results is set to a common size in the detailed display state and the enlarged display state, but the present invention is not limited to this. For example, the display sizes in the direction in which the plurality of analysis results are arranged may be different between the detailed display state and the enlarged display state.

In the above-described embodiment, an example is shown in which in the gel image display 94, “LM” and “UM”, which are indications indicating the reference values (lower limit and upper limit) corresponding to internal standard marker substances (LM and UM) are displayed on the scales 94a, in both the detailed display state and the enlarged display state, regardless of the degree of thinning of the number of displayed numerical values on the scales 94a, but the present invention is not limited to this. For example, only one of “LM” and “UM” may be displayed on the gel image display 94. Further, instead of character information indicating the reference value corresponding to the internal standard marker substance, a specific numerical value indicating the reference value may be displayed.

Further, in the above-described embodiment, an example is shown in which the number of displayed numerical values is thinned out such that the bias in the display intervals of the numerical values on the scales 94a in the gel image display 94 is reduced, but the present invention is not limited thereto. For example, the display intervals of the numerical values on the scales 94a may be biased. Note that when the enlarged display state is switched to the detailed display state, the displayed numerical values on the scales 94a may be directly thinned out, and the degree of thinning may be changed in a state where the bias is reduced such that numerical values different from the numerical values on the scales 94a displayed in the enlarged display state are displayed in the detailed display state.

Further, in the above-described embodiment, an example is shown in which the numerical values on the scales 94a are acquired based on the analysis of the reference sample, but the present invention is not limited to this. For example, preset values may be displayed on the scales 94a. Alternatively, the scales 94a may be set at regular intervals, and the displayed numerical values on the scales 94a may be set at regular display intervals. Further, the value on the scales 94a may be the timing (time) at which the peak of the measured waveform is detected for each component, instead of the numerical value indicating the size.

Further, in the above embodiment, an example is shown in which in the detailed display state, the analysis result check displays including the well position display 91, the measurement waveform display 92, the peak table 93, and the gel image display 94 are displayed in the result display area 52a of the display unit 52, but the present invention is not limited to this. For example, the analysis result check display in the detailed display state may not include one or any two out of the well position display 91, the measurement waveform display 92, and the peak table 93. Further, the plurality of analysis result check displays displayed in the detailed display state may include indications indicating measurement conditions, measurement order, and the like.

Further, in the above-described embodiment, an example is shown in which the control unit 54 of the analysis apparatus 102 acquires and analyzes the measurement value 111 output from the control unit 40 of the electrophoresis apparatus 101, but the present invention is not limited to this. For example, the analysis apparatus 102 may analyze the component of the object-to-be-measured separated by electrophoresis, by acquiring the measurement value 111 stored in advance in a storage device or storage medium such as the storage unit 53. That is, the measurement values 111 may be analyzed by the analysis apparatus 102 at a timing different from the acquisition of the measurement values 111 by the electrophoresis apparatus 101.

Further, in the above-described embodiment, an example is shown in which the chips 60a to 60c are provided with the preparation channel 63 for guiding the object-to-be-measured to the separation channel 62, but the present invention is not limited to this. For example, the chips 60a to 60c may be configured to have only the separation channel 62 without the preparation channel 63. Further, instead of a shape (cruciform) in which the separation channel 62 and the preparation channel 63 intersect so as to penetrate each other, the preparation channel 63 may intersect the separation channel 62 so as to form a T shape.

Further, in the above-described embodiment, an example is shown in which the electrophoresis apparatus 101 is configured to measure each of the plurality (three) of chips 60a to 60c, but the present invention is not limited to this. For example, one or two chips may be used to measure the object-to-be-measured, or four or more chips may be used. Further, even in a case where the electrophoresis apparatus 101 may be configured to measure each of the three chips 60a to 60c, only one or two chips may be designated (selected) for measurement.

Further, in the above-described embodiment, an example is shown in which the electrophoresis apparatus 101 is configured to perform microchip electrophoresis, but the present invention is not limited to this. For example, it may be configured to perform capillary electrophoresis without using a microchip.

Further, in the above-described embodiment, an example is shown in which the measurement value 111 of the object-to-be-measured is acquired by fluorescence detection, but the present invention is not limited to this. For example, the separated components of the object-to-be-measured may be detected by staining with a reagent.

Aspect

It will be appreciated by those skilled in the art that the exemplary embodiments described above are specific examples of the following aspects.

Item 1

An electrophoresis system including:

an electrophoresis apparatus including a measurement unit that measures an object-to-be-measured separated by electrophoresis in a channel including a separation channel for separating the object-to-be-measured;

an analysis apparatus that analyzes a component of the object-to-be-measured separated by electrophoresis, based on a measurement value of the object-to-be-measured that is measured by the measurement unit; and

a display unit that displays, in a result display area, a plurality of analysis result check displays including at least a gel image display showing a distribution of the components of the object-to-be-measured analyzed by the analysis apparatus, in which

the analysis apparatus is configured to switch between a detailed display state in which the plurality of analysis result check displays including at least the gel image display are displayed in the result display area of the display unit and an enlarged display state in which only the gel image display among the plurality of analysis result check displays is enlarged and displayed in the result display area of the display unit.

Item 2

The electrophoresis system according to Item 1, in which

the analysis apparatus is configured to reduce and thin out a number of displayed numerical values corresponding to scales in the gel image display such that the numerical values are not overlapped with each other, and make a degree of thinning of the number of displayed numerical values different from each other, in the detailed display state and the enlarged display state.

Item 3

The electrophoresis system according to Item 2, in which

the analysis apparatus is configured to display the numerical values in a common display size, in the detailed display state and the enlarged display state, and make the degree of thinning of the number of displayed numerical values different from each other such that the number of displayed numerical values in the enlarged display state is larger than in the detailed display state.

Item 4

The electrophoresis system according to Item 2 or 3, in which

the electrophoresis apparatus is configured to separate each of the plurality of objects-to-be-measured by electrophoresis, and

the analysis apparatus is configured to display, in the result display area of the display unit, the gel image display in which a plurality of analysis results showing the distribution of components of each of the plurality of objects-to-be-measured are displayed side by side, and set a display size in an arrangement direction of each of the plurality of analysis results to a common size in the detailed display state and the enlarged display state such that more analysis results are displayed in the enlarged display state than in the detailed display state.

Item 5

The electrophoresis system according to any one of Items 2 to 4, in which

the electrophoresis apparatus is configured to separate, by electrophoresis, the object-to-be-measured mixed with an internal standard marker substance that is a reference in the analysis of the component of the object-to-be-measured, and

the analysis apparatus is configured to display, in the gel image display, indications indicating a reference value corresponding to the internal standard marker substance, in both the detailed display state and the enlarged display state, regardless of the degree of thinning of the number of displayed numerical values.

Item 6

The electrophoresis system according to any one of Items 2 to 5, in which

the analysis apparatus is configured to, in a state where a bias in display intervals of the numerical values in the gel image display is reduced, make the degree of thinning of the number of displayed numerical values different in the detailed display state and the enlarged display state.

Item 7

The electrophoresis system according to any one of Items 2 to 6, in which

the electrophoresis apparatus is configured to separate, by electrophoresis, each of a reference sample that is the object-to-be-measured with known components and a sample-to-be-measured that is the object-to-be-measured with unknown components, and

the analysis apparatus is configured to acquire the numerical value based on analysis of the components of the reference sample, and make the degree of thinning of the number of displayed numerical values acquired based on the analysis of the components of the reference sample different from each other, in the detailed display state and the enlarged display state.

Item 8

The electrophoresis system according to any one of Items 1 to 7, in which

the electrophoresis apparatus is configured to separate each of the plurality of objects-to-be-measured by electrophoresis, and

the analysis apparatus is configured to, in the detailed display state, display the plurality of analysis result check displays including a measurement waveform display showing time-series values of the measurement values, a well position display showing a position of each of a plurality of wells in which each of the plurality of objects-to-be-measured is arranged, and the gel image display in which a plurality of analysis results showing a distribution of a component of each of the plurality of objects-to-be-measured are displayed side by side, in the result display area of the display unit, and in the enlarged display state, enlarge and display only the gel image display among the plurality of analysis result check displays, in the result display area of the display unit, without displaying the measurement waveform display and the well position display.

Item 9

The electrophoresis system according to Item 8, further including:

an operation unit that receives an input operation, in which

the analysis apparatus is configured, in the detailed display state, display the plurality of analysis result check displays including the gel image display in which the plurality of analysis results are displayed side by side, and the measurement waveform display corresponding to the one analysis result selected from among the plurality of analysis results in the gel image display based on the operation received by the operation unit, in the result display area of the display unit, and is configured to switch between the detailed display state and the enlarged display state, based on the operation received by the operation unit.

Item 10

An electrophoresis apparatus including:

a measurement unit that measures an object-to-be-measured separated by electrophoresis in a channel including a separation channel for separating the object-to-be-measured, in which

the electrophoresis apparatus is configured to switch between a detailed display state in which a plurality of analysis result check displays including at least a gel image display showing distribution of a component of the object-to-be-measured analyzed based on a measurement value of the object-to-be-measured that is measured by the measurement unit are displayed in a result display area of the display unit and an enlarged display state in which only the gel image display among the plurality of analysis result check displays is displayed in the result display area of the display unit.

Item 11

An electrophoresis analysis program causing a computer to execute:

a step of analyzing a component of an object-to-be-measured separated by electrophoresis, based on a measurement value obtained by measuring the object-to-be-measured separated by electrophoresis in a channel including a separation channel for separating the object-to-be-measured; and

a step of switching between a detailed display state in which a plurality of analysis result check displays including at least a gel image display showing a distribution of the analyzed component of the object-to-be-measured are displayed in a result display area of the display unit and an enlarged display state in which only the gel image display among the plurality of analysis result check displays is displayed in the result display area of the display unit.

Item 12

An electrophoresis analysis program causing a computer to execute:

a step of analyzing a component of an object-to-be-measured separated by electrophoresis, based on a measurement value obtained by measuring the object-to-be-measured separated by electrophoresis in a channel including a separation channel for separating the object-to-be-measured; and

a step of switching between a detailed display state in which the plurality of analysis result check displays including at least a gel image display showing a distribution of the analyzed component of the object-to-be-measured are displayed in the result display area of the display unit and an enlarged display state in which only the gel image display among the plurality of analysis result check displays is displayed in the result display area of the display unit.

What is claimed is:

1. An electrophoresis system comprising:
an electrophoresis apparatus including a measurement unit that measures an object-to-be-measured separated by electrophoresis in a channel including a separation channel for separating the object-to-be-measured;
an analysis apparatus that analyzes components of the object-to-be-measured separated by electrophoresis, based on a measurement value of the object-to-be-measured that is measured by the measurement unit; and
a display unit that displays, in a result display area, a plurality of analysis result check displays including at least a gel image display showing a distribution of the components of the object-to-be-measured analyzed by the analysis apparatus, wherein
the analysis apparatus is configured to switch between a detailed display state in which the plurality of analysis result check displays including at least the gel image display are displayed in the result display area of the display unit and an enlarged display state in which only the gel image display among the plurality of analysis result check displays is enlarged and displayed in the result display area of the display unit.

2. The electrophoresis system according to claim 1, wherein
the analysis apparatus is configured to reduce and thin out a number of displayed numerical values corresponding to scales in the gel image display such that the numerical values are not overlapped with each other, and make a degree of thinning of the number of displayed numerical values different from each other, in the detailed display state and the enlarged display state.

3. The electrophoresis system according to claim 2, wherein
the analysis apparatus is configured to display the numerical values in a common display size, in the detailed display state and the enlarged display state, and make the degree of thinning of the number of displayed numerical values different from each other such that the number of displayed numerical values in the enlarged display state is larger than in the detailed display state.

4. The electrophoresis system according to claim 2, wherein
the electrophoresis apparatus is configured to separate each of the plurality of objects-to-be-measured by electrophoresis, and
the analysis apparatus is configured to display, in the result display area of the display unit, the gel image display in which a plurality of analysis results showing the distribution of components of each of the plurality of objects-to-be-measured are displayed side by side, and set a display size in an arrangement direction of each of the plurality of analysis results to a common size in the detailed display state and the enlarged display state such that more analysis results are displayed in the enlarged display state than in the detailed display state.

5. The electrophoresis system according to claim 2, wherein
the electrophoresis apparatus is configured to separate, by electrophoresis, the object-to-be-measured mixed with an internal standard marker substance that is a reference in analysis of the component of the object-to-be-measured, and
the analysis apparatus is configured to display, in the gel image display, an indication indicating a reference value corresponding to the internal standard marker substance, in both the detailed display state and the enlarged display state, regardless of the degree of thinning of the number of displayed numerical values.

6. The electrophoresis system according to claim 2, wherein
the analysis apparatus is configured to, in a state where a bias in display intervals of the numerical values in the gel image display is reduced, make the degree of thinning of the number of displayed numerical values different in the detailed display state and the enlarged display state.

7. The electrophoresis system according to claim 2, wherein
the electrophoresis apparatus is configured to separate, by electrophoresis, each of a reference sample that is the object-to-be-measured with known components and a sample-to-be-measured that is the object-to-be-measured with unknown components, and
the analysis apparatus is configured to acquire the numerical values based on analysis of the components of the reference sample, and make the degree of thinning of the number of displayed numerical values acquired based on the analysis of the components of the reference sample different from each other, in the detailed display state and the enlarged display state.

8. The electrophoresis system according to claim 1, wherein
the electrophoresis apparatus is configured to separate each of the plurality of objects-to-be-measured by electrophoresis, and
the analysis apparatus is configured to, in the detailed display state, display the plurality of analysis result check displays including a measurement waveform display showing time-series values of the measurement values, a well position display showing a position of each of a plurality of wells in which each of the plurality of objects-to-be-measured is placed, and the gel image display in which a plurality of analysis results showing a distribution of a component of each of the plurality of objects-to-be-measured are displayed side by side, in the result display area of the display unit, and in the enlarged display state, enlarge and display only the gel image display among the plurality of analysis result check displays, in the result display area of the display unit, without displaying the measurement waveform display and the well position display.

9. The electrophoresis system according to claim 8, further comprising:
an operation unit that receives an input operation, wherein
the analysis apparatus is configured, in the detailed display state, display the plurality of analysis result check displays including the gel image display in which the plurality of analysis results are displayed side by side, and the measurement waveform display corresponding to the one analysis result selected from among the plurality of analysis results in the gel image display based on the operation received by the operation unit, in the result display area of the display unit, and is configured to switch between the detailed display state and the enlarged display state, based on the operation received by the operation unit.

10. An electrophoresis apparatus comprising:
a measurement unit that measures an object-to-be-measured separated by electrophoresis in a channel including a separation channel for separating the object-to-be-measured, wherein
the electrophoresis apparatus is configured to switch between a detailed display state in which a plurality of analysis result check displays including at least a gel image display showing a distribution of a component of the object-to-be-measured analyzed based on a measurement value of the object-to-be-measured that is measured by the measurement unit are displayed in a result display area of the display unit and an enlarged display state in which only the gel image display among the plurality of analysis result check displays is displayed in the result display area of the display unit.

11. An electrophoresis analysis method comprising:
a step of analyzing a component of an object-to-be-measured separated by electrophoresis, based on a measurement value obtained by measuring the object-to-be-measured separated by electrophoresis in a channel including a separation channel for separating the object-to-be-measured; and
a step of switching between a detailed display state in which a plurality of analysis result check displays including at least a gel image display showing a distribution of the analyzed component of the object-to-be-measured are displayed in a result display area of the display unit and an enlarged display state in which only the gel image display among the plurality of analysis result check displays is displayed in the result display area of the display unit.
